(19) United States
(12) Patent Application Publication
(10) Pub. No.: US 2007/0112430 A1
(43) Pub. Date: May 17, 2007

(54) ENDOPROSTHESIS FOR A SHOULDER JOINT

(76) Inventors: Beat R. Simmen, Tagelswangen (CH); Hans-Kaspar Schwyzer, Zuerich (CH); Marc Riner, Aristau (CH); Stephan Ingold, Gretzenbach (CH)

Correspondence Address: Knobble Martens Olson & Bear LLP, 14th Floor, 2040 Main Street, Irvine, CA, 92614, US

(21) Appl. No.: 11/460,098

(22) Filed: Jul. 26, 2006

(30) Foreign Application Priority Data
Nov. 16, 2005 (DE) 10 2005 054 649.8

(51) Int. Cl. A61F 2/40

(52) U.S. Cl. 623/19.14

(57) ABSTRACT

The present invention relates to an endoprosthesis for a shoulder joint having a stem member (10), extending axially along a first axis (100), with a distal end piece (11) insertable into the humerus of a patient and a proximal end piece (12), a stem head (20) being provided on the proximal end piece (12). In order to be able to achieve an anatomically optimum metaphyseal fit of the endoprosthesis in a simple way, according to the invention the stem member (10) and the stem head (20) are in each case constructed as modular components which are introducible into the patient's body separately from one another during implantation of the endoprosthesis, the stem member (10) having a connecting element (13) at its stem-head-side proximal end (12) and the stem head (20) having at its humeral, distal end (22) a distal connecting element (23) correspondingly constructed so as to be complementary thereto, which connecting elements can be brought into engagement with one another and in the implanted state of the endoprosthesis co-operate with one another so that a force-based connection is formed between stem member (10) and stem head (20).

The present invention relates to an endoprosthesis for a shoulder joint, the endoprosthesis having the following: a stem member, extending axially along a first axis, which stem member has a distal end piece insertable into the humerus of a patient and a proximal end piece on which a stem head is provided; an intermediate piece having at its humeral end a connecting element which can be brought into engagement with an intermediate-piece-side proximal connecting element constructed on the stem head so as to be complementary thereto, so that in the implanted state of the endoprosthesis the intermediate piece extends along a second axis different from the direction of the first axis; and a ball head, receivable in a glenoid cavity, which is connectible by way of the intermediate piece to the stem head of the stem member.

Such an endoprosthesis for a shoulder joint is known in principle from medical technology. For example, DE 299 18 589 U1 discloses an endoprosthesis having a stem member which has an axial stem shaft for implantation in the humerus and a stem head. Furthermore, in the known endoprosthesis there is provided a head calotte which acts as ball head and which is connected by means of an alignment piece or intermediate piece to the stem head of the stem member, the intermediate piece being so constructed that the direction and position of the calotte axis can be adjusted in respect of inclination and rotation.

Furthermore, a shoulder joint prosthesis having a stem member is disclosed in FR 2 773 469, there likewise being provided a swivellable intermediate piece or alignment piece that carries a spherical calotte. In that prosthesis, a hemispherical cavity having a threaded bore is provided at the metaphyseal end of the stem member. The cavity and threaded bore are in alignment with a neck axis (second axis), the angle of which relative to the stem axis (first axis) has already been established.

In detail, in such a joint prosthesis known from the prior art, the swivellable intermediate piece is accommodated in the cavity, the intermediate piece having at its humeral end a semi-spherical surface concentric with the semi-spherical surface of the cavity. Furthermore, the intermediate piece has at its other end a tapered surface which is constructed so as to be eccentric with respect to the sphere centre point common to both spherical surfaces. On that tapered surface there is seated a head calotte having a corresponding tapered recess. The axis of that recess is arranged eccentrically with respect to the centre point of the calotte. It is also provided that the centres of the spherical surfaces of the cavity in the stem member, of the inner and outer semi-spherical surfaces on the intermediate piece and of the spherical surface of the screw head lie in a common central point when the screw has been tightened. As a result, the intermediate piece is swivellable in all directions relative to the stem member and is at the same time rotatable. By adjusting the inclination and rotation with respect to the stem axis with the aid of a manipulating head, it is possible to set the neck axis parallel to the optimum neck axis.

Such so-called third-generation shoulder joint endoprostheses known from medical technology already allow relatively good reconstruction of the shoulder joint, because in each case an intermediate piece is provided by means of which it is possible, to a certain extent, to adjust the dorso-medial offset of the centre point of the humeral head relative to the centre point of the stem member and the inclination of the humeral head. In particular, in such third-generation endoprostheses, the soft-tissue tension can be adjusted, for example by means of different ball head radii and ball head heights, so that it is possible to influence the stability of the treated joint. However, the adaptation of the endoprosthesis achieved by means of different ball head radii and ball head heights can have the result that the ball head segment no longer corresponds to the original and so the anatomy is not correctly reconstructed.

Because the success of a shoulder prosthesis depends substantially on the re-establishment of the muscular equilibrium of the shoulder musculature, it is important that by the use of the endoprosthesis the anatomically correct production of the shape of the bony constituents and accordingly of the joint geometry is achieved in the best possible way. In the case of the third-generation shoulder prostheses described above, it is to a certain extent already possible—by virtue of their adaptability achievable with the aid of the intermediate piece—to configure the prosthesis more individually. A disadvantage of the solutions known from the prior art, however, is that it has frequently not been possible hitherto to achieve an anatomically optimum metaphyseal fit. As already mentioned, although the inclination and the dorso-medial offset can be influenced to a certain extent by manipulation of the intermediate piece it is often the case that the angular position of the intermediate piece cannot be set in the optimum starting position, because the known endoprostheses do not have sufficient degrees of freedom in respect of their being matched to the anatomy of the patient. In particular, the adjustment of the direction and position of the calotte axis (second axis) is difficult to achieve in the prior art.

Starting on that basis, the problem underlying the present invention is to develop an endoprosthesis for a shoulder joint of the kind mentioned at the beginning to the effect that it is possible to obtain the best possible match of the endoprosthesis to the anatomy of the patient. A particular objective is to simplify the adjustment of the direction and position of the calotte axis (second axis) in comparison with the prior art.

That problem is solved according to the invention by a shoulder joint endoprosthesis of the kind mentioned at the beginning wherein the stem member and the stem head are in each case constructed as modular components which are introducible into the patient's body separately from one another during implantation of the endoprosthesis, the stem member having a connecting element at its stem-head-side proximal end and the stem head having at its humeral, distal end a distal connecting element correspondingly constructed so as to be complementary thereto, which connecting elements can be brought into engagement with one another and in the implanted state of the endoprosthesis co-operate with one another so that a force-based connection can be made between stem member and stem head.

The solution according to the invention has many considerable advantages over the endoprosthesis known from the prior art and mentioned above: by virtue of the modular structure of the stem member and the stem head, it is possible, during implantation of the endoprosthesis, for first the stem member to be fixated in the diaphyseal humerus and only then for the proximal corpus, constructed as the stem head, to be positioned on the proximal end of the already implanted stem member. On the one hand, this simplifies the implantation of the stem member in the humerus, because the stem member is a relatively small and flexibly usable component in comparison with the stem members known from the prior art.

On the other hand, in comparison with the conventional solutions, the modular structure of the stem head makes it better and more easily possible to achieve an anatomically optimum metaphyseal fit. In particular, by suitable selection of the corpus height or the height of the stem head, the anatomically correct overall height of the “stem member/stem head” complex can be set in the optimum way, while an optimum match to the length and thickness of the humerus is obtained by suitable selection of the size of the stem member, especially in respect of length and diameter.

In the present invention, the first axis, along which the stem member extends, is to be understood as being the longitudinal axis of the stem member. The term “second axis” is to be understood as being the calotte axis. In the present specification, the term “modular component” is used to indicate components of the endoprosthesis that are separately constructed and introducible into the patient's body where they are then joined together to form the final endoprosthesis. In particular, a “modular component” is a functional component of the endoprosthesis. By virtue of the modular structure of the stem member and the stem head, not only are the individual components of the endoprothesis accordingly smaller and easier to handle, which facilitates implantation, but also the functions associated with the components in question are divided; this allows optimum matching of the endoprosthesis to the anatomy of the patient, because each functional component can be matched individually in the optimum way. For example, the stem member assumes primarily the function of fixation and force introduction in the patient's humerus, whereas the stem head is used to divert the forces transmitted from the ball head by way of the intermediate piece to the stem member.

By dividing the stem member and stem head into two functionally independent components it is also possible for each component to be matched to the anatomy of the patient in the best possible way in respect of its function. For example, the modular structure of the stem member allows it to be used in the optimum way in the metaphyseal portion of the humerus. Because loosening of prosthesis components is a complication that frequently arises in shoulder endoprosthetics, the solution according to the invention offers a possible way of implanting the stem member with sufficient stability in order thus to prevent premature loosening of the implanted endoprosthesis.

Advantageous further developments of the endoprosthesis according to the invention are given in the subsidiary claims.

For example, in an advantageous embodiment the connection formed by way of the connecting element at the proximal end of the stem member and the connecting element at the distal (humeral) end of the stem head is so configured that, on implantation of the endoprosthesis, the stem member and the stem head can be rotated relative to one another about the first axis. The advantage of that development is clear: because the stem member and stem head can be rotated relative to one another about the first axis (stem axis), a further degree of freedom, namely the degree of freedom about the stem axis, is provided. It is thus easier to achieve optimum reconstruction of the original anatomy. In particular, during the operation, the relative angular position between stem head and stem member can be set by rotation of those components, which are constructed as modular components, relative to one another about the stem axis. In other words, by means of the advantageous development, it is accordingly possible to set the angular position of the second axis in respect of the first axis. This is a considerable advantage, because the alignment of the second axis (calotte axis) in respect of the first axis (stem axis) can be adjusted as desired by rotation of the stem head in an especially simple and easily implemented way. In particular, that positioning or adjustment is also possible when the stem member is already firmly fixated in the humerus of the patient. The solution according to the invention thus provides not only improved matching of the endoprosthesis to the anatomy of the patient but also a simplification of the adjustment of the direction and position of the calotte axis in respect of the stem axis.

In order to ensure that, after the adjustment and establishment of the direction and position of the calotte axis relative to the first axis (stem axis), a rigid and, especially, rotationally secure connection can be made between stem head and stem member, in a preferred further development of the embodiments of the endoprosthesis according to the invention mentioned above, the stem head has a locking means which, in a state where the connecting element at the proximal end of the stem member and the connecting element at the distal (humeral) end of the stem head are joined and the rotated position of the stem head relative to the stem member (which is, for example, already firmly implanted) has been established, co-operates with the stem member as a result of an external manipulation, so that the connection formed by way of the connecting element at the proximal end of the stem member and the connecting element at the distal end of the stem head is fixed.

In a preferred realisation of the last-mentioned embodiment, in which the stem head has a locking means, the locking means is a screw which extends through a through-hole provided in the stem head along the first axis and which is received in a thread provided at the proximal end of the stem member. Such a solution is particularly simple to implement and at the same time easy to operate, because the through-hole provided in the stem head along the first axis can readily be accessed by the surgeon, so that the rigid and, especially, rotationally secure connection between the stem member and stem head that is achievable by means of the locking means can be achieved in a simpler way. It will be understood, however, that here other embodiments of the locking means are also conceivable. For example, it is also possible to provide a plurality of screw connections. Fixing the stem member and stem head relative to one another with the aid of pins or clips is also conceivable.

With a view to the connection formed by way of the connecting element at the proximal end of the stem member and the connecting element at the distal end of the stem head, in an advantageous embodiment it is also provided that the connecting element at the proximal end of the stem member and the connecting element at the distal end of the stem head are constructed in the form of elements, especially tapered elements, that are insertable one inside the other, the direction of insertion corresponding substantially to the direction of the first axis (stem axis). This is a preferred solution by which the stem member and stem head can be joined together, while at the same time freely selectable rotational positioning is retained by means of the connection between the corpus (stem head) and stem member. Because the direction of insertion corresponds substantially to the direction of the first axis (stem axis), it is possible to connect the stem head to the stem member in a simpler way. Accordingly, the solution according to the invention provides not only a further degree of freedom, which allows optimum matching of the endoprosthesis to the anatomy of the patient, but also simpler implantation of the stem member and stem head in the humerus of the patient. Here too, it will be understood that other solutions for the connection between stem member and stem head are conceivable.

In order to achieve the best possible rotational stability of the stem member to be installed in the humerus, in an advantageous further development of the endoprosthesis according to the invention the stem member and/or the stem head have, at least to some extent, rectangular cross-sectional shaping, in which case it would be possible to use as stem member a bi-conical stem pin having rectangular cross-sectional shaping. A bi-conical stem allows cementless fixation of the stem member in the metaphyseal portion of the humerus. In the systems known from the prior art, cementless fixation assumes a press-fit of the prosthesis in the spongy proximal bone. Accordingly, there is no direct contact between the implant and the cortical bone, which can often result in insufficient stability of the “bone-implant” bond. The use of eccentric ball heads, however, renders rotational stability ever more important, especially in the case of cementless systems. By the configuration of the stem member according to the invention as a cementless bi-conical stem, a primary stability is achieved, the preferably rectangular cross-section of the stem member providing the necessary rotational stability. It would be possible, for example, for the stem member to be based on the geometry of diaphyseal fixation which has already been used and become established in hip prosthetics. The rectangular cross-sectional shaping is not, however, limited only to the stem member. Rather, the stem head too can, if necessary, have such cross-sectional shaping in order, for example, to achieve as stepless as possible a transition at the “stem member/stem head” connection.

In an advantageous realisation of the last-mentioned embodiment, in which the stem member and/or the stem head have, at least to some extent, rectangular cross-sectional shaping, the stem member has a cross-section that tapers from the proximal end to the distal end. This is advantageous especially in respect of the fixation of the stem member in the humerus. In particular, it is possible for the cross-section of the stem member to taper bi-conically from proximal to distal. Accordingly, the long-term rotational stability of the implanted endoprosthesis can be ensured in the optimum way. It will be understood, however, that other solutions are also conceivable in respect of the cross-sectional shaping of the stem member or stem head.

In order to facilitate the best possible fixing of the direction and position of the second axis (articulation axis or calotte axis), in a further advantageous embodiment in respect of the stem head the point of intersection between the first axis (stem axis) and the second axis (calotte axis) lies in the interior of the stem head, it being possible to obtain the anatomically optimum position of the stem member, which is dependent upon the patient in question, by suitable selection of the distance between the point of intersection and the proximal end of the stem member. Accordingly, the solution according to the invention facilitates not only freely selectable rotational positioning between stem member and stem head, so that the position of the second axis (calotte axis) is adjustable in respect of rotation relative to the stem axis, but also the inclination of the axis direction of the second axis. In particular, it is thus possible to achieve better matching of the endoprosthesis to the anatomy of the patient in question.

In a preferred realisation of the last-mentioned embodiment, by suitable selection of the length of a distal (humeral-side) region of the stem head extending in the direction of the first axis it is possible to adjust the distance between the point of intersection and the proximal end of the stem member and accordingly to obtain the anatomically optimum length of the stem member. By virtue of the modular structure of the humerus implant it is accordingly possible, by the use of a proximal corpus (stem head) the length of the distal region of which is matched individually to the anatomy of the patient, to obtain an anatomically optimum metaphyseal fit of the endoprosthesis.

In a preferred further development, albeit known in part from the prior art, for setting an angle of inclination between the ball head and the glenoid cavity the intermediate piece is constructed so as to be rotatable and swivellable relative to the first and second axis. The ball head is preferably fixed to that intermediate piece, for example by means of a tapered connection. The ball head can have different radii, heights and eccentricities in order to achieve an optimum anatomical reposition. By constructing the intermediate piece as such an inclination element, the inclination and rotation with respect to the first axis (stem axis) can accordingly be adjusted in the optimum way. This is achieved by the intermediate piece's being swivellable in all directions relative to the stem member and at the same time rotatable.

In a further solution, which is likewise known in part from the prior art, the intermediate piece is constructed so as to be longitudinally displaceable along the calotte axis (second axis) in order to adjust the distance between the stem member and the ball head. As a result, exact positioning of the dorso-medial offset is possible. By means of the solution according to the invention, consisting of separately constructed modular components which can be combined with one another, an endoprosthesis is accordingly obtained with which the original anatomy can be better reconstructed. In particular, by means of the solution according to the invention the dorso-medial offset of the centre point of the humeral head relative to the centre point of the stem member, the inclination of the humeral head and the retrotorsion position of the humeral head relative to the stem axis are taken into account, this being ensured by the possibility of making adjustments between the individual modular components and by the additional degree of freedom about the first axis achieved by the modular structure. Furthermore, a plurality of ball head, stem head and stem member sizes is advantageously available in order to be able to match the endoprosthesis as individually as possible. Preferably, the ball heads are rounded at the edges, so that they can be better matched to the resection surface and the tubercula and, furthermore, do not endanger the soft tissue sliding over them.

In a further preferred development of the endoprosthesis according to the invention, fastening means, especially through-holes and/or threaded holes, are provided on the stem head and/or stem member for fastening implantation material, such as, for example, suture material or bone screws. For example, soft tissue can be attached to the fastening means. It is also possible to provide the corpus (stem head) with threaded holes for the fixing of bone screws or screws which fix bone plates. It will be understood that other embodiments of the fastening means are also conceivable here.

Preferably, the stem member and/or the stem head consist(s) of titanium or a titanium alloy, but other body-compatible metals are also conceivable here, such as, for example, a cobalt/chromium alloy. Furthermore, it is also possible for high-performance ceramics, for example, to be used at least partly for the individual components of the endoprosthesis. A further possibility in respect of the ball head material is a titanium forging alloy which has been treated, for example, with titanium-niobium. Such a surface finish provides a high degree of wear-resistance. As material for the individual components of the endoprosthesis, however, there also comes into consideration, for example, a stainless iron forging alloy, for example according to DIN ISO 5832/1. High-performance ceramics, such as, for example, aluminium oxide ceramics AI 203 according to DIN ISO 6474 are also conceivable. Such ceramics are bio-inert, bio-compatible and corrosion-resistant and have excellent fracture toughness combined with extreme hardness.

It is also preferable for the surfaces of the individual prosthesis components to be surface-treated for the purpose of better bony integration. In particular, it is advantageous to roughen the surfaces partially or completely, for example using a suitable blasting process, so that an optimum bone/implant bond is achievable. It is also possible, for the purpose of better bony integration, to coat the surface partially or completely with hydroxyl apatite or similar calcium phosphate compounds. It will be understood, however, that other materials and other surface treatments are conceivable for that purpose.

The invention will be described in greater detail below with reference to an embodiment shown diagrammatically in the Figures.

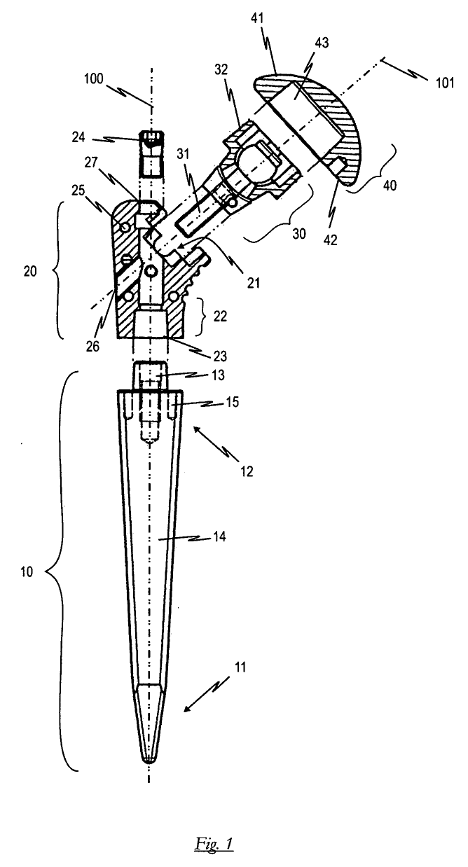

FIG. 1 is an exploded sectional view of a preferred exemplary embodiment of the endoprosthesis according to the invention;

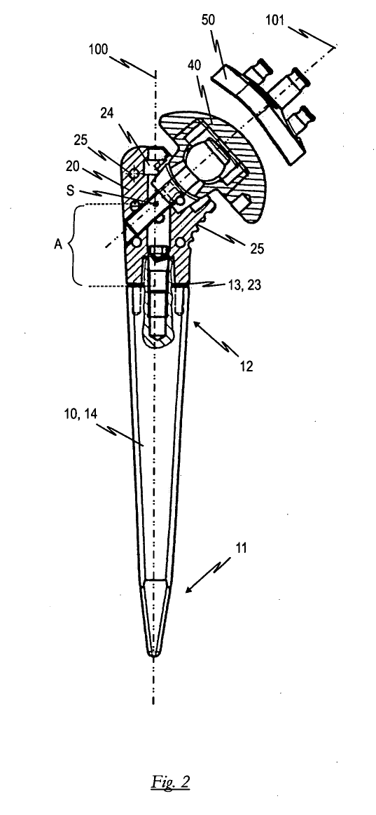

FIG. 2 is a sectional view of the endoprosthesis according to FIG. 1 in the assembled state;

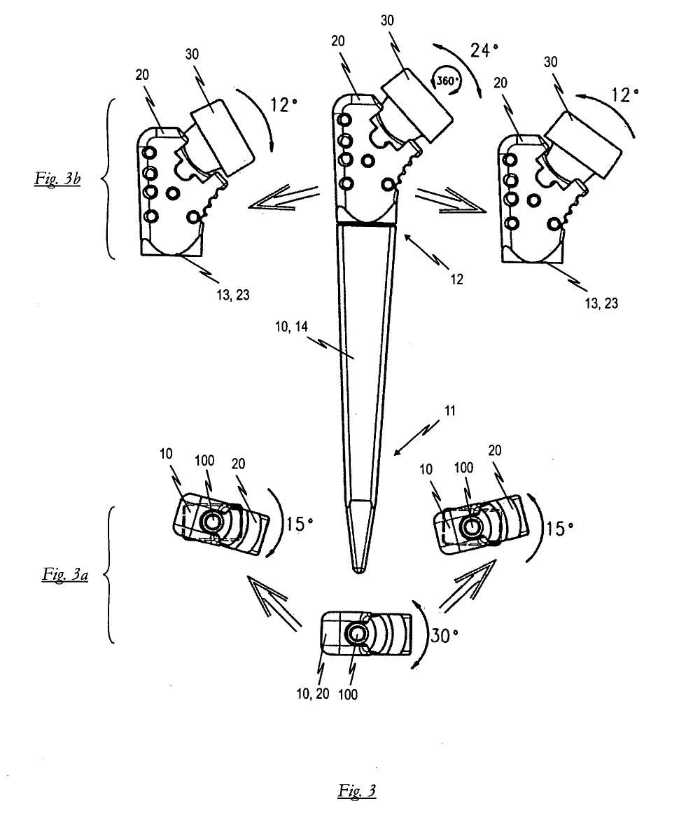

FIG. 3a is a plan view of the stem head of the endoprosthesis according to FIG. 1 in different angular positions; and

FIG. 3b is a side view of the stem head of the endoprosthesis according to FIG. 1 at different angles of inclination.

The embodiment according to the invention of the endoprosthesis for a shoulder joint according to the invention in accordance with FIGS. 1 and 2 has a stem member 10 having a stem shaft 14 along a stem axis 100 (first axis) and a distal and proximal end 11, 12. At the proximal end 12 of the stem shaft 14 there is provided a device 15 for installation of the stem shaft 14 in the humerus bone. That device can also be used for removing the stem shaft from the bone. Furthermore, at the proximal end 12 of the stem shaft 14 there is provided a connecting element 13 in the form of a tapered pin.

The endoprosthesis according to the preferred embodiment also has a stem head 20 constructed separately from the stem member 10. At the humerus-side, distal end 22 of the stem head 20 there is provided a distal connecting element 23, constructed so as to be complementary to the connecting element 13, in the form of a tapered recess. In the assembled state, as shown in FIG. 2, the distal connecting element 23 of the stem head 20 is in engagement with the proximal-side connecting element 13 of the stem member 10, so that the stem head 20 and the stem member 10 can be joined together by a force-based connection. In detail, the humeral-side connecting element 23, which is constructed in the form of a tapered recess, of the stem head 20 is formed along the stem axis 100 (first axis), so that the connecting element 13 at the proximal end of the stem member 10 and the connecting element 23 at the distal end 22 of the stem head 20 are insertable one inside the other, the direction of insertion corresponding substantially to the direction of the stem axis 100.

Furthermore, on the stem head 20 there are provided fastening means 25, especially through-holes or threaded holes, to which implantation material, such as, for example, suture material or bone screws, and also soft tissue, can be attached.

Furthermore, on the stem head 20 there is provided a concave, truncated-cone-shaped articulation surface 21, the axis 101 of which is parallel to the calotte axis, here at an angle of about 135° to the stem axis 100, and is referred to as the “second axis” hereinbelow. Axially relative to that second axis 101, a bore 26 having an internal thread is provided at the base of the articulation surface 21. The articulation surface 21 serves as an intermediate-piece-side proximal connecting element which can be brought into engagement with a stem-head-side or humeral-side distal connecting element 31 of an intermediate piece 30 constructed so as to be complementary thereto.

At the proximal end of the stem head 20 there is further provided a bore 27 into which a locking means, such as, for example, a nut 24 can be introduced in order to lock the stem head 20 with the stem member 10 so as to be rotationally secure. Advantageously, the bore 27 extends along the stem axis 100, so that on implantation of the endoprosthesis the locking means 24 can readily be accessed by the surgeon and so the stem head 20 and the stem member 10 can be rigidly joined together.

An intermediate piece 30 is insertable into the truncated-cone-shaped articulation surface 21 provided in the stem head 20. As already indicated, for that purpose the intermediate piece 30 has a stem-head-side or humeral-side distal connecting element 31, which can be brought into engagement with the intermediate-piece-side proximal connecting element 21 of the stem head 20 which is constructed so as to be complementary thereto, so that in the implanted state of the endoprosthesis the intermediate piece 30 extends along the second axis 101 (calotte axis). In the preferred embodiment of the endoprosthesis according to the invention, the intermediate piece 30 is constructed as an inclination element, so that during the implantation of the endoprosthesis the second axis (calotte axis) 101 can be matched, in respect of the stem axis 100, to the anatomy of the patient in the best possible way. Furthermore, the intermediate piece 30 is so constructed that the length of the intermediate piece 30 is adjustable to a certain extent in order to set the distance between the stem member 10 and the ball head 40.

The ball head 40 is positioned on the proximal end of the intermediate piece 30. Because the intermediate piece 30 allows a rotational and swivelling movement about the second axis 101, the alignment of the ball head 40 with the natural joint surface of a glenoid or with an implanted glenoid element 50 (see FIG. 2) is adjustable.

The ball head 40 is advantageously a spherical segment similar to the spherical segment of the osteotomised joint. In an idealised calotte, the articulation surface of the calotte 40 lies on a radius around its sphere centre point. The calotte 40 has a flat underside 42 opposite from the spherical surface 41, and it can be provided that the plane of the underside 42 intersects geometrically the theoretical spherical surface in a circle. The calofte axis or second axis 101 is determined by the centre point of that circle and the sphere centre point.

Eccentrically to the second axis 101 there is provided in the underside 42 a tapered depression 43, the tapered wall of which, constructed axially about the ball head axis 101, and the depth of which are matched to the proximal end 32 of the intermediate piece 30. As already stated, the spherical surface 41 of the ball head 40 is provided to co-operate with the natural joint surface of the glenoid or with an implanted glenoid element 50.

The ball head 40 is positioned on the intermediate piece 30, the calotte 40 having the depression 43 being pressed onto an outer tapered surface 32 of the intermediate piece 30 and accordingly fixed in place. By virtue of the eccentric arrangement of the ball head 40, the sphere centre point of the joint can be matched to the optimum anatomical conditions by rotation of the intermediate piece 30 about the second axis 101. Once the dorso-medial offset and the ball head inclination have been established, the intermediate piece can be fixed in a conventional way.

FIG. 3b shows how the angle of inclination can be adjusted with the aid of the intermediate piece 30. In the preferred embodiment of the endoprosthesis according to the invention, the inclination of the intermediate piece or of the ball head arranged at the proximal end of the intermediate piece (not shown explicitly in FIG. 3b) is adjustable in an angle range of ±12° about the direction of the second axis 101 predetermined by the stem head 20.

The adjustment of the dorso-medial offset, the centre point of the humeral head with respect to the centre point of the stem, the inclination of the humeral head and the retrotorsion position of the humeral head with respect to the stem axis 100, which is facilitated by suitable adjustment of the intermediate piece 30, is known from the prior art and will not be described in greater detail here.

The solution according to the invention is distinguished especially by the fact that the stem member 10 and the stem head 20 are in the form of modular components which are constructed separately from one another. It can thus be ensured, firstly, that the surgeon has particularly manageable components to hand during the operation, which components can be implanted more easily. The assembly of the endoprosthesis is effected during implantation of the individual components. In particular, for example, not only the stem component 10 and the stem head 20 can be implanted separately from one another, but the entire intermediate piece 30 is insertable in a disassembled state.

In addition to simpler implantation of the endoprosthesis, the solution according to the invention also offers a further degree of freedom, however, whereby optimum anatomical reposition can be achieved. In particular, the stem member 10 and the stem head 20 are constructed so as to be rotatable about the first axis 100 (stem axis), which was not possible in any of the solutions known from the prior art. By means of the chosen connection between corpus 20 (stem head) and stem member 10, in particular the rotational position can be freely selectable. By the use of different heights of stem head, the anatomically optimum overall height of the “stem member/stem head” is obtainable, the stem head 20 being so constructed that the point of intersection S (see FIG. 2) between the first axis 100 and the second axis 101 lies in the interior of the stem head 20, so that by suitable selection of the distance A between the point of intersection S and the proximal end 12 of the stem member 10 it is possible to obtain an anatomically optimum length of stem member 10 dependent upon the patient in question. For example, it would be possible, by suitable selection of the length of a distal region 22 of the stem head 20 extending in the direction of the first axis 100, to adjust the distance A between the point of intersection S and the proximal end 12 of the stem member 10 and accordingly to obtain the anatomically optimum length of the stem member 10.

In the case of the advantageous embodiment of the endoprosthesis according to the invention described herein, the stem member 10 and the corpus 20 (stem head) to be constructed thereon each have a rectangular cross-section which tapers bi-conically from proximal to distal, it being possible to achieve optimum matching to the length and thickness of the humerus bone by suitable selection of the stem size (length and diameter). Such a construction of the stem cross-section enables optimum rotational stability in the humerus to be achieved.

In order to achieve the best possible stability of the treated joint, and in order to be able to establish the optimum possible soft tissue tension, in the solution according to the invention it is also possible for ball heads 40 having different radii, heights and eccentricities to be provided in order to achieve an anatomical reposition.

It should be pointed out that the implementation of the invention is not limited to the exemplary embodiment described in the Figures but is also possible in a plurality of variants.

1-14. (canceled)

15. An endoprosthesis for a shoulder joint, comprising:
a stem member extending along a first axis and having a distal end insertable into a humerus bone of a patient and a proximal end opposite the distal end;
a stem head removably coupleable to the proximal end of the stem member, the stem head defining a second axis different from the first axis, the second axis intersecting the first axis at an intersection point;
an intermediate member removably coupleable to the stem head, the intermediate member extending along the second axis; and
a ball head removably coupleable to the intermediate member, the ball head configured to be received into a glenoid cavity,
wherein the stem member and stem head are introducible into the patient's body separately from one another during implantation of the endoprosthesis, the stem member and stem head coupleable to each other via a friction force.

16. The endoprosthesis of claim 15, wherein the stem member and stem head are coupleable to each other via a press-fit connection.

17. The endoprosthesis of claim 15, wherein the stem member and stem head are coupleable such that the stem member and stem head can be rotated relative to one another about the first axis on implantation of the endoprosthesis.

18. The endoprosthesis of claim 15, further comprising a locking mechanism configured to form a substantially rigid connection between the stem member and the stem head.

19. The endoprosthesis of claim 18, wherein the locking mechanism comprises a screw extendible through a bore in the stem head and coupleable to the proximal end of the stem member.

20. The endoprosthesis of claim 15, wherein the stem member has a tapered element and the stem head has a tapered recess, the tapered element configured to form a press-fit connection with the tapered recess to couple the stem member with the stem head.

21. The endoprosthesis of claim 15, wherein at least one of the stem member and the stem head has a rectangular cross-section.

22. The endoprosthesis of claim 15, wherein the stem member tapers from the proximal end to the distal end.

23. The endoprosthesis of claim 15, wherein the intersection point lies on the stem head and a distance is defined between the intersection point and the proximal end of the stem member, such that an anatomically optimum length of the stem member can be obtained for a particular patient by selecting a stem head defining a suitable distance.

24. The endoprosthesis of claim 15, wherein the intermediate member is configured to be rotatable and swivellable relative to the first and second axes to define an angle of inclination between the ball head and the glenoid cavity.

25. The endoprosthesis of claim 15, wherein the intermediate member is longitudinally displaceable along the second axis so as to adjust a distance between the stem member and the ball head.

26. The endoprosthesis of claim 15, wherein at least one of the stem member and the stem head comprises titanium or a titanium alloy.

27. The endoprosthesis of claim 15, wherein a surface of at least one of the stem member and stem head is configured to enhance bone integration.

28. The enoprosthesis of claim 27, wherein said surface is roughened.

29. The endoprosthesis of claim 27, wherein said surface is coated with hydroxyl apatite or similar calcium phosphate compounds.

30. A method for implanting an endoprosthesis for a shoulder joint, comprising:
implanting a stem member within a diaphyseal humerus bone of a patient, the stem member extending along a first axis; and
coupling a stem head to a proximal end of the implanted stem member, the stem head defining a second axis different from the first axis, the second axis intersecting the second axis.

31. The method of claim 30, further comprising adjusting the angular position of the second axis relative to the first axis by rotating the stem head relative to the stem member.

32. The method of claim 30, wherein the stem member is implanted in a cementless manner.

33. The method of claim 30, further comprising selecting a stem head having a height configured to achieve an anatomically optimum length of the stem member for a patient.

34. The method of claim 30, further comprising aligning a ball head coupled to the stem head with a joint surface of an implanted glenoid element.
